(19) United States
(12) Patent Application Publication
(10) Pub. No.: US 2019/0059827 A1
(43) Pub. Date: Feb. 28, 2019

(54) SYSTEM AND METHOD FOR IMAGING A SUBJECT

(71) Applicant: GENERAL ELECTRIC COMPANY, Schenectady, NY (US)

(72) Inventors: KATELYN ROSE NYE, GLENDALE, WI (US); GIREESHA RAO, PEWAUKEE, WI (US); JOHN SABOL, SUSSEX, WI (US); JOHN TKACZYK, DELANSON, NY (US)

(73) Assignee: GENERAL ELECTRIC COMPANY, Schenectady, NY (US)

(21) Appl. No.: 15/685,294

(22) Filed: Aug. 24, 2017

(51) Int. Cl. A61B 6/02; A61B 6/00; A61B 6/04; G06T 11/00

(52) U.S. Cl. CPC A61B 6/025; A61B 6/4035; A61B 6/4208; G06T 11/005; G06T 11/008; A61B 6/0407

(57) ABSTRACT

A system for imaging a subject is provided. The system includes a radiation source, a radiation detector, and a controller. The radiation source is operative to transmit electromagnetic rays through the subject while the radiation source travels along a path defined by a sweep angle that is less than 365 degrees. The radiation detector is operative to receive the electromagnetic rays after having passed through the subject. The controller is operative to: acquire preliminary data regarding the subject from a sensor; determine an imaging parameter from the preliminary data; and acquire one or more images of the subject via the radiation source and the radiation detector based at least in part on the imaging parameter.

### BACKGROUND

### Technical Field

Embodiments of the invention relate generally to medical technologies, and more specifically, to a system and method for imaging a subject.

### Discussion of Art

Digital tomosynthesis is an imaging technology that provides for volume data acquisition from selected regions of a body. Many tomosynthesis systems include a mobile arm that moves a radiation source along a curved and/or linear path with respect to a subject such that a plurality of projections of a body part are obtained. A digital processor then reconstructs a three dimensional (“3D”) image/model of the subject from the projections. Unlike traditional computer tomography (“CT”), which involves the reconstruction of a 3D image from projections that form a complete circumference around the subject, the projections utilized in tomosynthesis typically form a partial circumference, i.e., an arc, as opposed to a full circle. Moreover, many tomosynthesis systems only move/sweep the radiation source along the path once during a scan. Accordingly, the acquisition parameters of many tomosynthesis systems must be tightly controlled during a scanning procedure in order to mitigate the risk of artifacts and/or other imaging errors.

Standard acquisition parameters often include sweep angle, sweep direction, patient barrier-object distance, number of projections, and/or total radiation dose. Potential acquisition-related artifacts may include blurring-ripple, ghost artifact-distortion, poor spatial resolution, image noise, and/or metallic artifact indicators. A comprehensive understanding of the relationships between acquisition parameters and potential associated artifacts is often critical to optimizing an acquisition technique and avoiding misinterpretation of artifacts. For example, sweep direction may be chosen on the basis of the anatomy of interest and the purpose of the examination so as to reduce the influence of blurring-ripple, ghost artifact-distortion, and/or metallic artifacts. In such scenarios, a bone fracture may be extended in one predominate direction transverse to the bone axis so that the sweep direction is parallel to the bone axis direction. Alternately, a sweep direction may be relative to the axis of metal rods or screws used to stabilize a bone fracture so that metal artifacts are minimized. Adjusting the sweep angle, number of projections, and/or radiation dose will usually optimize depth resolution, noise level, avoid ripple in the sections of interest, and/or reduce unnecessary radiation exposure without compromising image quality. Adjusting the source-to-detector distance may change the magnification of anatomy and the field of view in the X-ray image whereby more or less of the anatomy will appear in the image.

Therefore, in many tomosynthesis systems, it is important that the radiologist and technologist operating the system follow appropriate protocols for different examination types and/or subject specific contingencies in order to sufficiently capture the anatomical features targeted and/or to mitigate the risk of incurring artifacts in the final image set. Operators of tomosynthesis systems, e.g., radiologists and technologists, however, may be unfamiliar with the proper techniques for performing tomosynthesis acquisitions over varied anatomy and/or patient sizes. Further, many traditional tomosynthesis systems may not provide guidance to operators with respect to adjusting/tuning the parameters of a tomosynthesis system to a particular subject for a particular type of acquisition.

What is needed, therefore, is an improved system and method for imaging a subject.

### BRIEF DESCRIPTION

In an embodiment, a system for imaging a subject is provided. The system includes a radiation source, a radiation detector, and a controller. The radiation source is operative to transmit electromagnetic rays through the subject while the radiation source travels along a path defined by a sweep angle that is less than 365 degrees. The radiation detector is operative to receive the electromagnetic rays after having passed through the subject. The controller is operative to: acquire preliminary data regarding the subject from a sensor; determine an imaging parameter from the preliminary data; and acquire one or more images of the subject via the radiation source and the radiation detector based at least in part on the imaging parameter.

In another embodiment, a method for imaging a subject is provided. The method includes acquiring preliminary data regarding the subject via a sensor; and determining an imaging parameter from the preliminary data via a controller. The method further includes acquiring one or more images of the subject via a radiation source and a radiation detector based at least in part on the imaging parameter. The radiation detector receives electromagnetic rays transmitted though the subject by the radiation source while the radiation source travels along a path defined by a sweep angle that is less than 365 degrees.

In yet another embodiment, a non-transitory computer readable medium storing instructions is provided. The stored instructions are configured to adapt a controller to acquire preliminary data regarding a subject via a sensor, and to determine an imaging parameter from the preliminary data. The stored instructions are further configured to adapt the controller to acquire one or more images of the subject via a radiation source and a radiation detector based at least in part on the imaging parameter. The radiation detector receives electromagnetic rays transmitted though the subject by the radiation source while the radiation source travels along a path defined by a sweep angle that is less than 365 degrees.

### DRAWINGS

The present invention will be better understood from reading the following description of non-limiting embodiments, with reference to the attached drawings, wherein below:

FIG. 1 is a schematic diagram of a system for imaging a subject, in accordance with an embodiment of the invention;

FIG. 2 is a schematic diagram of another orientation of the system of FIG. 1, in accordance with an embodiment of the invention;

FIG. 3 is a schematic diagram of yet another orientation of the system of FIG. 1, in accordance with an embodiment of the invention;

FIG. 4 is a schematic diagram of still yet another orientation of the system of FIG. 1, in accordance with an embodiment of the invention;

FIG. 5 is a schematic diagram of still yet another orientation of the system of FIG. 1, in accordance with an embodiment of the invention;

FIG. 6 is a schematic diagram of still yet another orientation of the system of FIG. 1, in accordance with an embodiment of the invention; and

FIG. 7 is a flow chart depicting a method for imaging a subject utilizing the system of FIG. 1, in accordance with an embodiment of the invention.

### DETAILED DESCRIPTION

Reference will be made below in detail to exemplary embodiments of the invention, examples of which are illustrated in the accompanying drawings. Wherever possible, the same reference characters used throughout the drawings refer to the same or like parts, without duplicative description.

As used herein, the terms “substantially,” “generally,” and “about” indicate conditions within reasonably achievable manufacturing and assembly tolerances, relative to ideal desired conditions suitable for achieving the functional purpose of a component or assembly. As used herein, “electrically coupled,” “electrically connected,” and “electrical communication” mean that the referenced elements are directly or indirectly connected such that an electrical current may flow from one to the other. The connection may include a direct conductive connection, i.e., without an intervening capacitive, inductive or active element, an inductive connection, a capacitive connection, and/or any other suitable electrical connection. Intervening components may be present. The term “real-time,” as used herein, means a level of processing responsiveness that a user senses as sufficiently immediate or that enables the processor to keep up with an external process. As further used herein, the terms “scan,” “procedure,” and/or “imaging procedure” refer to the acquisition of data by an imaging system from which one or more images of a subject may be generated from. The term “imaging parameter,” as used herein, means a setting of a device or a property of a subject to be imaged that affects the operation of an imaging system.

Additionally, while the embodiments disclosed herein are described with respect to an x-ray based imaging system, e.g., a tomosynthesis imaging system, it is to be understood that embodiments of the present invention are equally applicable to other devices and/or imaging systems which preform tomography, have low tolerances for parameter settings, and/or have difficult to calculate parameters. Further, embodiments of the present invention related imaging systems may be used to analyze objects within any material which can be internally imaged, generally. As such, embodiments of the present invention are not limited to analyzing objects within human tissue.

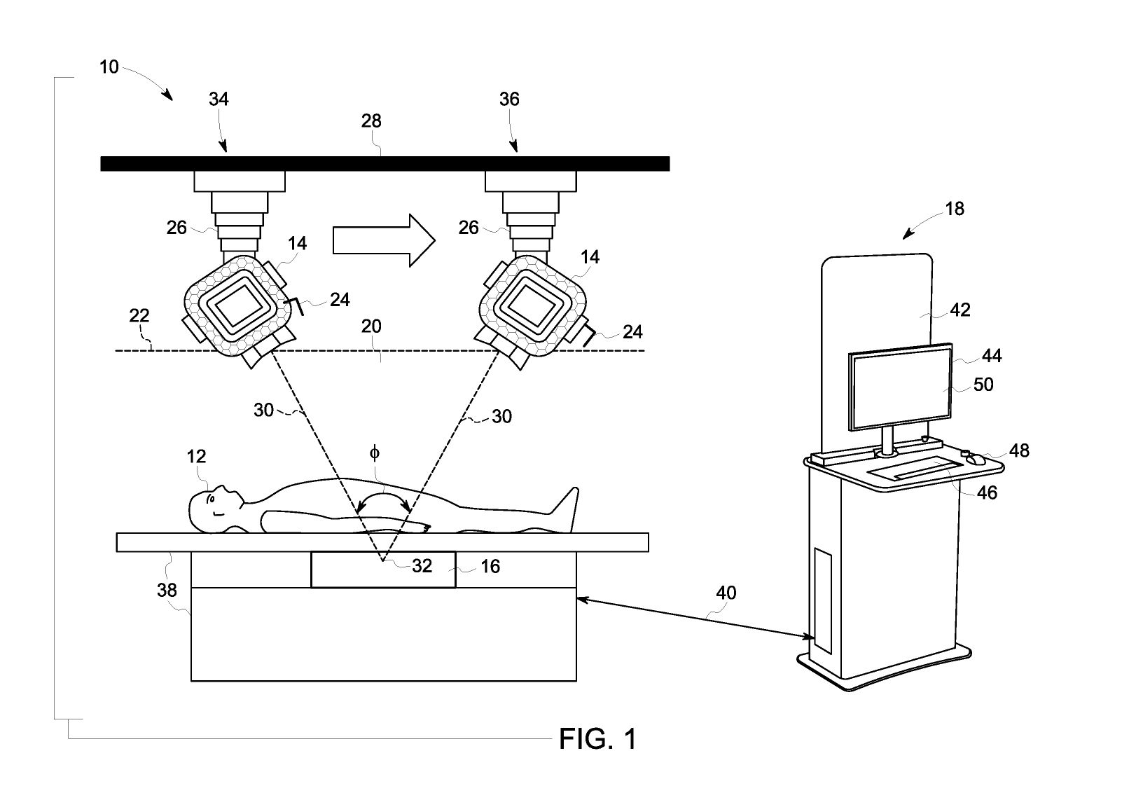

Referring now to FIG. 1, the major components of a system 10 for imaging a subject/object/patient 12, in accordance with an embodiment of the invention, are shown. The system 10 includes a radiation source/device 14, a radiation detector 16, and a controller 18. The radiation source 14 is operative to transmit electromagnetic rays/radiation 20 through the subject 12 while the radiation source 14 travels along a path 22 defined by a sweep angle Ø. The radiation detector 16 is operative to receive the electromagnetic rays 20 after having passed through the subject 12. As will be appreciated, and explained in greater detail below, the controller 18 is operative to acquire preliminary data regarding the subject 12 from a sensor 24, determine an imaging parameter from the preliminary data, and to acquire one or more images of the subject 12 based at least in part on the imaging parameter.

Accordingly, as shown in FIG. 1, the radiation source 14 may be rotatably mounted to a mobile arm 26 secured to a support structure 28, e.g., a mount and/or the ceiling of a room, such that the radiation source 14 is able to train the electromagnetic rays 20 along a line of projection 30 that continuously intersects a target location 32 on the radiation detector 16 as the mobile arm 26 moves the radiation source 14 along the path 22. The path 22 may have a start 34 position and an end/stop position 36 such that the line of projection 30 sweeps an area of the subject 12 defined by the sweep angle Ø. As will be appreciated, while the path 22 is shown herein as being linear, it will be understood that, in other embodiments, the path 22 may have a curved shape and/or any other shape configured for tomosynthesis. Further, the sweep angle Ø may be less than 365°, and in some embodiments, may be between about 0° to 180°, 20° to 100°, 20° to 80°, 20° to 40°, or 20° to 30°. As will be appreciated, in some embodiments, the sweep angle Ø may be greater than or equal to 365°. Further still, While the radiation rays 20 are discussed herein as being x-rays, it is to be understood that the radiation source 14 may emit other types of electromagnetic rays, e.g., radio waves, visible light, ultra-violet light, gamma rays, etc., which can be used to image the subject 12.

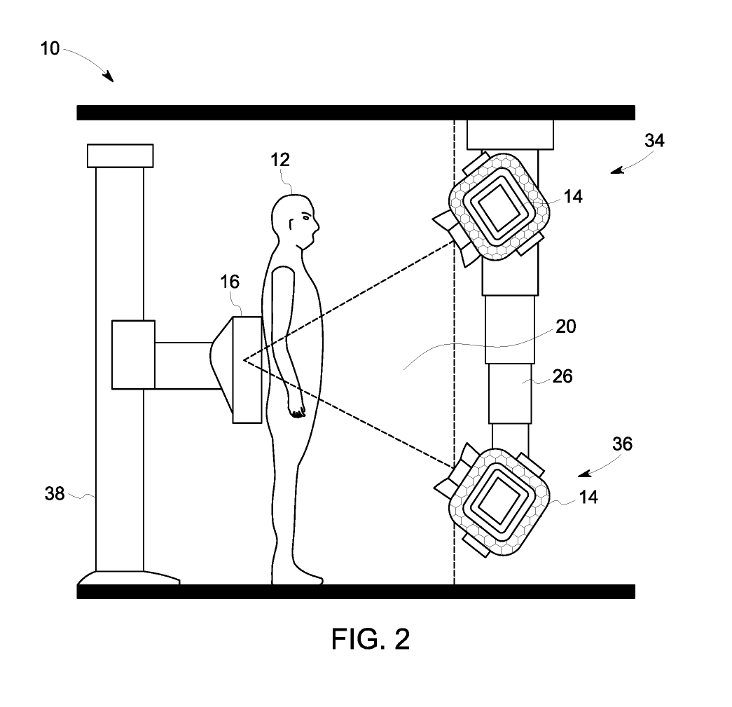

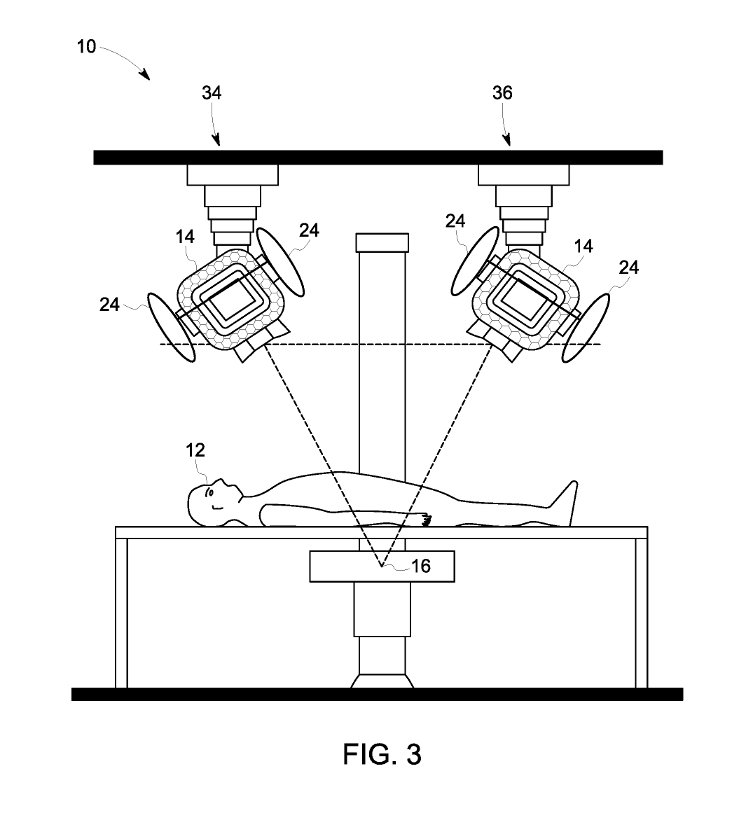

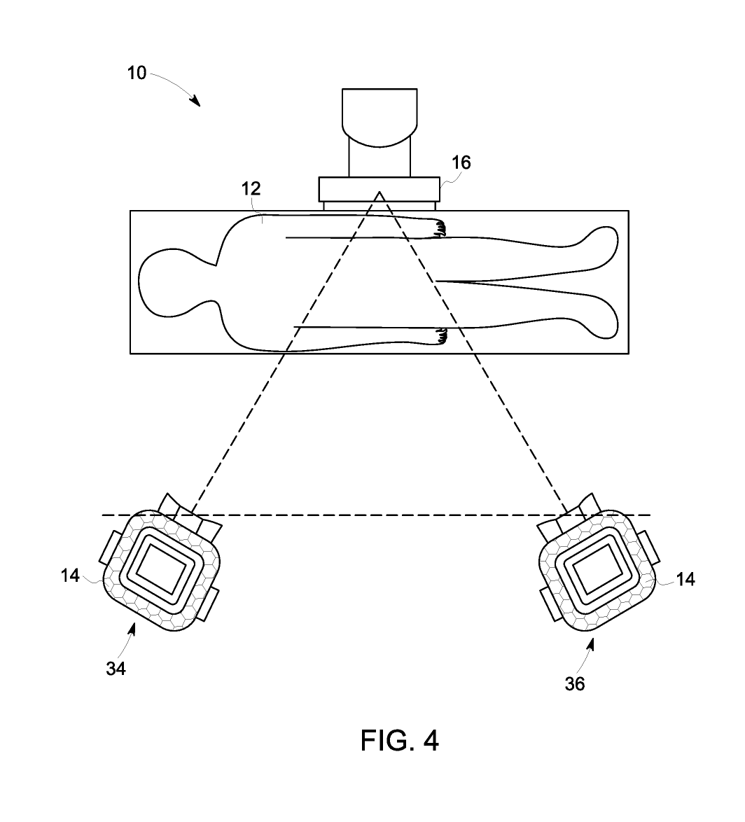

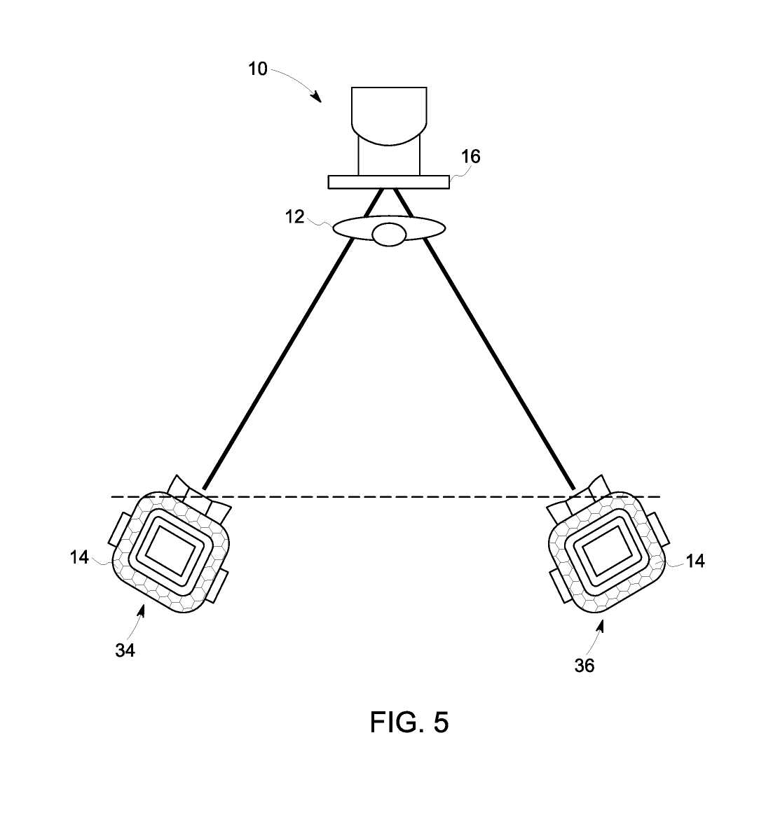

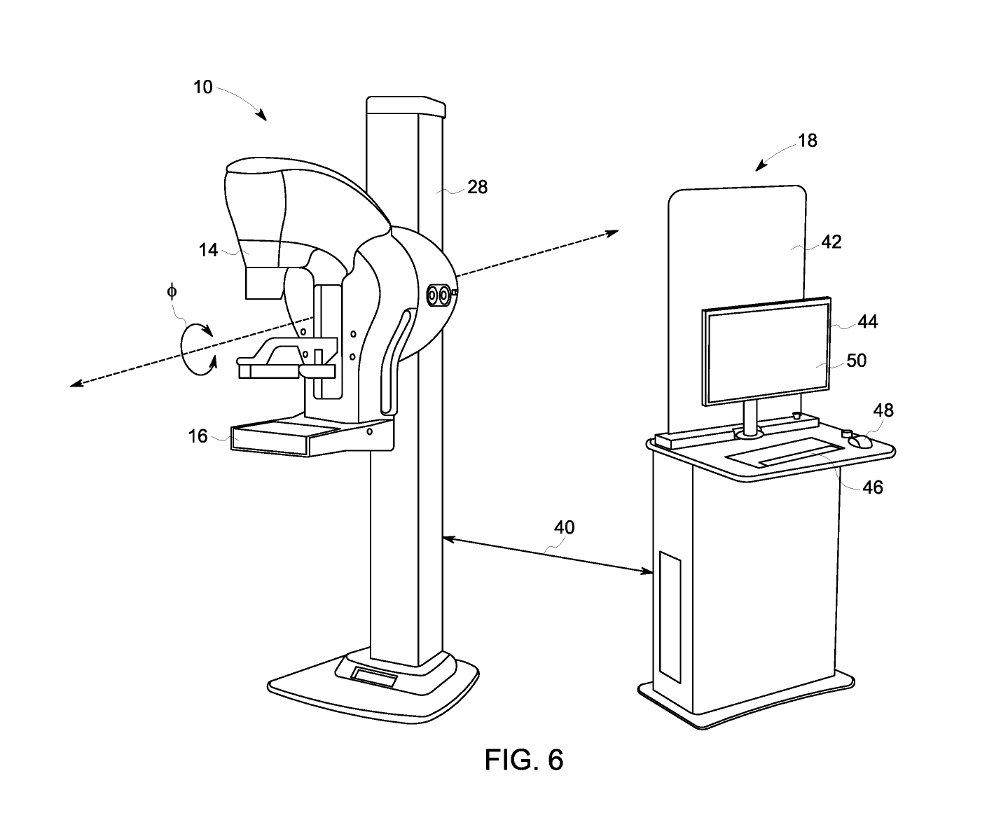

As further shown in FIG. 1, the radiation detector 16 is positioned opposite the radiation source 14 such that the subject 12 is disposed between the radiation source 14 and the radiation detector 16. While the radiation detector 16 is depicted herein as being stationary with respect to the subject 12, it will be understood, that, in other embodiments, the radiation detector 16 may move in relation to the subject 12. Additionally, the radiation detector 16 may be integrated into a subject support structure 38, e.g., a table and/or other platform structure which, in embodiments, may be operative to support the entire subject 12 or a part of the subject 12. For example, as shown in FIGS. 1-6, in embodiments, the system 10 may be configured to perform a table horizontal sweep (FIG. 1) for supine imaging, a wallstand vertical sweep (FIG. 2) for upright imaging, a wallstand horizontal sweep (FIG. 3) for supine imaging, a wallstand cross-table sweep for cross-table imaging of a patient laying down (FIG. 4) and/or standing (FIG. 5); and/or a mammography sweep (FIG. 6).

The controller 18 may be a workstation having at least one processor and a memory device as shown in FIG. 1 or, in other embodiments, the controller 18 may be embedded/integrated into one or more of the various components of the system 10 disclosed above. In embodiments, the controller 18 may be in electrical communication with the radiation source 14, radiation detector 16, and/or the sensor 24 via an electrical communication connection 40. The connection 40 may be a wired and/or wireless connection. As will be appreciated, in embodiments, the controller 18 may include a radiation shield 42 that protects an operator of the system 10 from the radiation rays 20 emitted by the radiation source 14. The controller 18 may further include a display 44, a keyboard 46, mouse 48 and/or other appropriate user input devices, that facilitate control of the system 10 via a user interface 50. Data regarding the radiation rays 20 received by the radiation detector 16 may be electrically communicated to the controller 18 from the radiation detector 16 via cable/electronic connection 40 such that the controller 18 generates/reconstructs one or more images which may be shown on the display 44.

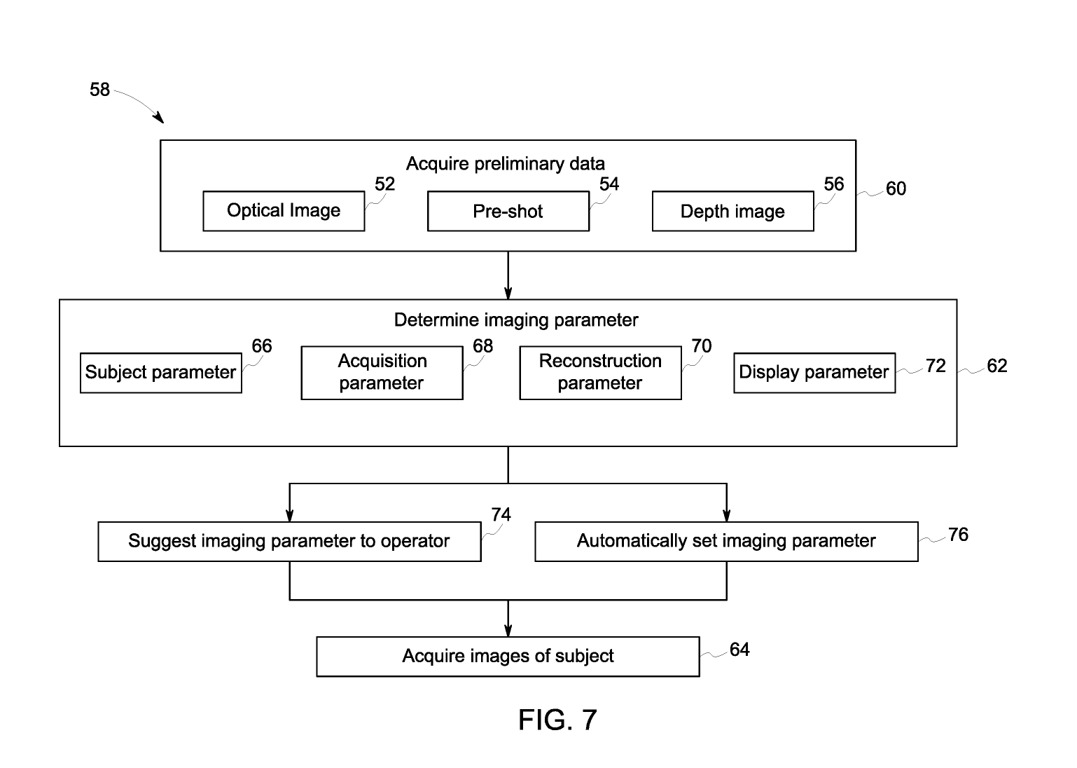

As stated above, the sensor 24 is operative to acquire preliminary data from the subject 12, which is then used by the controller 18 to determine/calculate one or more imaging parameters of the system 10. Accordingly, in embodiments, the sensor 24 may be an optical camera as shown in FIG. 1, which acquires an image/picture of the subject 12, i.e., the preliminary data is an optical image 52 (FIG. 7). As such, the sensor 24 may be mounted on the radiation source 14, e.g., a radiation tube, on the mobile arm 26, support structure 28, and/or in any other manner so as to provide clear access, e.g., a line of sight, from the sensor 24 to the subject 12. As will be appreciated, in such embodiments, the sensor 24 may be operative to image the subject 12 with visible, infrared, ultra-violet, and/or other forms of electromagnetic radiation suitable for imaging the subject 12. Further, the sensor 24 may acquire a single image and/or a plurality of images.

In embodiments, the sensor 24 may include the radiation source 14 and the radiation detector 16 as shown in FIG. 2, i.e., the preliminary data is a pre-shot 54 (FIG. 7), which, as used herein, means an image of a subject acquired by an imaging system and analyzed prior to the imaging system acquiring subsequent images of the subject. For example, in an embodiment, the pre-shot may be a low resolution image acquired via a lower radiation dose than images which are subsequently acquired via the radiation source 14 and detector 16 and used to make a medical diagnosis. Additionally, the pre-shot may include multiple views of the subject 12.

The sensor 24 may also be a depth camera as shown in FIG. 3, wherein the sensor 24 may include two or more stereo cameras having lasers, e.g., light imaging, detection, and ranging (“LIDAR”), such that the preliminary data is a depth image 56 (FIG. 7) of the subject 12.

As will be appreciated, in embodiments, the sensor 24 may be a scale that measures the weight and/or mass of the subject 12. For example, the sensor 24 may be able to determine the distribution of the subject's 12 weight and/or mass on the support structure 38, e.g., table. Thus, as will be understood, the sensor 24 may be a non-ionizing sensor.

In certain aspects, the preliminary data may come from outside the system 10. For example, in embodiments, the preliminary data may be a radiology medical image, e.g., an x-ray, digital tomosynthesis, magnetic resonance image (“MRI”), positron emission tomographic (“PET”) image, and/or any other type of medical image, acquired by a different imaging system, or by the same imaging system at a different time, and saved in a database accessible to the controller 18. Similarly, the controller 18 may access additional data concerning the subject 12, e.g., patient medical histories stored in a database external to the room in which the system 10 is housed.

Further, in certain aspects, an artificial intelligence (“AI”) and/or deep learning algorithm may be utilized to process and/or obtain the preliminary data. For example, in embodiments, such an algorithm may generate/obtain the preliminary data by analyzing medical information, to include pre-acquired images, pulled from a database, as described above.

Further still, in embodiments, an RFID tag, and/or optical barcode, may be disposed on the subject 12 which is read into the system 10 via an input device, e.g., a scanner, such that controller 18 may query one or more external databases for information regarding the subject 12 using the RFID tag, and/or barcode. For example, such information may specify a particular anatomical region as the target of the scan, i.e., the target site/region of interest.

Turning now to FIG. 7, a method 58 for imaging the subject 12 utilizing the system 10 is shown. The method 58 includes acquiring 60 the preliminary data via the sensor 24, determining 62 the imaging parameters from the preliminary data, and acquiring 64 images of the subject 12 based at least in part on the imaging parameters. In embodiments, the method may further include suggesting/conveying 74 the imaging parameters to an operator and/or automatically setting 76, i.e., “auto-set”, the imaging parameters.

In embodiments, determining 62 the imaging parameters may be based on one or more machine learning algorithms, to include deep learning algorithms and/or population health averages. As will be understood, in some embodiments, the imaging parameters may be determined 62 based on input received by the controller 18 via the keyboard 46, mouse 48, or other suitable input device, e.g., a touch screen. For example, the system 10 may acquire and show an optical image 52 of the subject 12 on the display 44, and an operator of the system 10 may then select a portion of the subject 12 in the image, which in turn, may be used by the controller 18 to adjust one or more of the imaging parameters disclosed herein.

In embodiments wherein the sensor 24 acquires an image, optical and/or pre-shot, determining 62 the imaging parameter may include segmentation of the image and/or classification of the segments thereof. For example, the controller 18 may provide for the image segments to be identifies/labeled as being associated with the subject and/or a particular part thereof, e.g., a wrist, left hand, knee, etc.

As further shown in FIG. 7, the imaging parameters may be/include a subject parameter 66, an acquisition parameter 68, a reconstruction parameter 70, a display parameter 72, and/or any other suitable type of imaging parameter, e.g., a PAC Push option, e.g., a specific type of image reconstruction. The subject parameter 66 may include: subject positional data; subject size data, e.g., adult/pediatric, small/medium/large; an anatomy type; and/or a view type. The acquisition parameter 68 may include: killovoltage (kV), milliampers (mA), and/or exposure time data, e.g., static or dynamic accounting for varying patient thickness; radiation dose ratio data; collimation data; path data, e.g., direction and/or speed/acceleration of the radiation source 14 along the path 22; the number of projections to be reconstructed; a pivot point along the path 22; and/or filter information. The reconstruction parameter 70 may include: imaging processing data, e.g., soft tissue, bone, or metal implants; reconstruction algorithms, e.g., iterative reconstructions and/or back projection; scatter correction data; start and stop reconstruction heights; slice interval; slice orientation; sampling factor, e.g., slab thickness, and/or image look. The display parameter 72 may include: a gray-scale brightness level, a range of brightness window, a look-up-table (LUT) transform of the brightness, a first slice to display indicator, i.e., an indicator which identifies the first slice from the reconstruction stack that is to be shown on screen, e.g., the first, last, middle, and/or a user defined slice; and/or the reconstruction type/model/algorithm to be used by the controller 18.

For example, in embodiments where the preliminary data is a pre-shot 54, the controller 18 may determine the orientation of an internal object, e.g., a long bone, within the subject 12, and accordingly, automatically 76 adjust/configure the path 22 such that the radiation source 14 moves in a manner, e.g., direction, speed, acceleration, etc., that minimizes the potential for artifacts. In some embodiments, the controller 18, based on the pre-shot, may determine that the path 22 of the system 10 cannot be adjusted/configured to mitigate the possibility of artifacts and, in turn, suggests 74 to the operator a new position for the subject 12 for which the path 22 may be adjusted/configured to mitigate the possibility of artifacts.

Suggesting 74 the imaging parameter may be facilitated via the display 44/interface 50 and/or via an audio signal and/or message. For example, in embodiments, the controller 18 may cause one or more pop-up windows to appear on the display 44 that contain recommended/suggested imaging parameters derived from the preliminary data acquired from subject 12 via the sensor 24 and/or from an external database.

Finally, it is also to be understood that the imaging system 10 may include the necessary electronics, software, memory, storage, databases, firmware, logic/state machines, microprocessors, communication links, displays or other visual or audio user interfaces, printing devices, and any other input/output interfaces to perform the functions described herein and/or to achieve the results described herein, which may be accomplished in real-time. For example, as previously mentioned, the system may include at least one processor and system memory/data storage structures, which may include random access memory (RAM) and read-only memory (ROM). The at least one processor of the system may include one or more conventional microprocessors and one or more supplementary co-processors such as math co-processors or the like. The data storage structures discussed herein may include an appropriate combination of magnetic, optical and/or semiconductor memory, and may include, for example, RAM, ROM, flash drive, an optical disc such as a compact disc and/or a hard disk or drive.

Additionally, a software application that adapts the controller to perform the methods disclosed herein may be read into a main memory of the at least one processor from a computer-readable medium. The term “computer-readable medium,” as used herein, refers to any medium that provides or participates in providing instructions to the at least one processor of the system 10 (or any other processor of a device described herein) for execution. Such a medium may take many forms, including but not limited to, non-volatile media and volatile media. Non-volatile media include, for example, optical, magnetic, or opto-magnetic disks, such as memory. Volatile media include dynamic random access memory (DRAM), which typically constitutes the main memory. Common forms of computer-readable media include, for example, a floppy disk, a flexible disk, hard disk, magnetic tape, any other magnetic medium, a CD-ROM, DVD, any other optical medium, a RAM, a PROM, an EPROM or EEPROM (electronically erasable programmable read-only memory), a FLASH-EEPROM, any other memory chip or cartridge, or any other medium from which a computer can read.

While in embodiments, the execution of sequences of instructions in the software application causes at least one processor to perform the methods/processes described herein, hard-wired circuitry may be used in place of, or in combination with, software instructions for implementation of the methods/processes of the present invention. Therefore, embodiments of the present invention are not limited to any specific combination of hardware and/or software.

It is further to be understood that the above description is intended to be illustrative, and not restrictive. For example, the above-described embodiments (and/or aspects thereof) may be used in combination with each other. Additionally, many modifications may be made to adapt a particular situation or material to the teachings of the invention without departing from its scope.

For example, in an embodiment, a system for imaging a subject is provided. The system includes a radiation source, a radiation detector, and a controller. The radiation source is operative to transmit electromagnetic rays through the subject while the radiation source travels along a path defined by a sweep angle that is less than 365 degrees. The radiation detector is operative to receive the electromagnetic rays after having passed through the subject. The controller is operative to: acquire preliminary data regarding the subject from a sensor; determine an imaging parameter from the preliminary data; and acquire one or more images of the subject via the radiation source and the radiation detector based at least in part on the imaging parameter. In certain embodiments, the sensor is an optical camera and the preliminary data is an optical image. In certain embodiments, the sensor includes the radiation source and the radiation detector and the preliminary data is a pre-shot. In certain embodiments, the sensor is a depth camera and the preliminary data is a depth image. In certain embodiments, the imaging parameter is at least one of a subject parameter, an acquisition parameter, a reconstruction parameter, and a display parameter. In certain embodiments, the path is configured for tomosynthesis. In certain embodiments, the electromagnetic rays are x-rays. In certain embodiments, the controller is further operative to auto-set the imaging parameter. In certain embodiments, the controller conveys the imaging parameter to an operator for manual adjustment of one or more components of the system that correspond to the imaging parameter.

Other embodiments provide for a method for imaging a subject. The method includes acquiring preliminary data regarding the subject via a sensor; and determining an imaging parameter from the preliminary data via a controller. The method further includes acquiring one or more images of the subject via a radiation source and a radiation detector based at least in part on the imaging parameter. The radiation detector receives electromagnetic rays transmitted though the subject by the radiation source while the radiation source travels along a path defined by a sweep angle that is less than 365 degrees. In certain embodiments, the sensor is an optical camera, and acquiring preliminary data regarding the subject via a sensor includes acquiring an optical image of the subject via the optical camera. In certain embodiments, the sensor includes the radiation source and the radiation detector, and acquiring preliminary data regarding the subject via a sensor includes acquiring a pre-shot of the subject via the radiation source and the radiation detector. In certain embodiments, the sensor is a depth camera, and acquiring preliminary data regarding the subject via a sensor includes acquiring a depth image of the subject via the depth camera. In certain embodiments, the imaging parameter is at least one of a subject parameter, an acquisition parameter, a reconstruction parameter, and a display parameter. In certain embodiments, the path is configured for tomosynthesis. In certain embodiments, the electromagnetic rays are x-rays.

Yet still other embodiments provide for a non-transitory computer readable medium storing instructions. The stored instructions are configured to adapt a controller to acquire preliminary data regarding a subject via a sensor, and to determine an imaging parameter from the preliminary data. The stored instructions are further configured to adapt the controller to acquire one or more images of the subject via a radiation source and a radiation detector based at least in part on the imaging parameter. The radiation detector receives electromagnetic rays transmitted though the subject by the radiation source while the radiation source travels along a path defined by a sweep angle that is less than 365 degrees. In certain embodiments, the sensor is an optical camera and the preliminary data is an optical image. In certain embodiments, the sensor includes the radiation source and the radiation detector and the preliminary data is a pre-shot. In certain embodiments, the imaging parameter is at least one of a subject parameter, an acquisition parameter, a reconstruction parameter, and a display parameter.

Accordingly, as will be appreciated, by using a sensor to acquire preliminary data of a subject prior to committing to a set of imaging parameters for a given imaging procedure/acquisition, some embodiments of the present invention reduce the risk that low image quality, missed anatomy and/or artifacts will result from an operators' unfamiliarity with tomosynthesis, and/or other similar imaging techniques. Thus, some embodiments of the invention provide for improved accuracy, e.g., fewer artifacts, and/or for a reduced radiation exposure to the subject over traditional radiation based imaging systems. Moreover, by suggesting and/or automatically setting imaging parameters, some embodiments may reduce the amount of operator intervention and/or reimaging of the subject, which in turn may greatly simplify the work process for operators of tomosynthesis and similar imaging systems.

Additionally, while the dimensions and types of materials described herein are intended to define the parameters of the invention, they are by no means limiting and are exemplary embodiments. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, terms such as “first,” “second,” “third,” “upper,” “lower,” “bottom,” “top,” etc. are used merely as labels, and are not intended to impose numerical or positional requirements on their objects. Further, the limitations of the following claims are not written in means-plus-function format are not intended to be interpreted as such, unless and until such claim limitations expressly use the phrase “means for” followed by a statement of function void of further structure.

This written description uses examples to disclose several embodiments of the invention, including the best mode, and also to enable one of ordinary skill in the art to practice the embodiments of invention, including making and using any devices or systems and performing any incorporated methods. The patentable scope of the invention is defined by the claims, and may include other examples that occur to one of ordinary skill in the art. Such other examples are intended to be within the scope of the claims if they have structural elements that do not differ from the literal language of the claims, or if they include equivalent structural elements with insubstantial differences from the literal languages of the claims.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property.

Since certain changes may be made in the above-described invention, without departing from the spirit and scope of the invention herein involved, it is intended that all of the subject matter of the above description shown in the accompanying drawings shall be interpreted merely as examples illustrating the inventive concept herein and shall not be construed as limiting the invention.

What is claimed is:

1. A system for imaging a subject comprising:
a radiation source operative to transmit electromagnetic rays through the subject while the radiation source travels along a path defined by a sweep angle that is less than 365 degrees;
a radiation detector operative to receive the electromagnetic rays after having passed through the subject; and
a controller operative to:
acquire preliminary data regarding the subject from a sensor;
determine an imaging parameter from the preliminary data; and
acquire one or more images of the subject via the radiation source and the radiation detector based at least in part on the imaging parameter.

2. The system of claim 1, wherein the sensor is an optical camera and the preliminary data is an optical image.

3. The system of claim 1, wherein the sensor includes the radiation source and the radiation detector and the preliminary data is a pre-shot.

4. The system of claim 1, wherein the sensor is a depth camera and the preliminary data is a depth image.

5. The system of claim 1, wherein the imaging parameter is at least one of a subject parameter, an acquisition parameter, a reconstruction parameter, and a display parameter.

6. The system of claim 1, wherein the path is configured for tomosynthesis.

7. The system of claim 1, wherein the electromagnetic rays are x-rays.

8. The system of claim 1, wherein the controller is further operative to auto-set the imaging parameter.

9. The system of claim 1, wherein the controller conveys the imaging parameter to an operator for manual adjustment of one or more components of the system that correspond to the imaging parameter.

10. A method for imaging a subject comprising:
acquiring preliminary data regarding the subject via a sensor;
determining an imaging parameter from the preliminary data via a controller;
acquiring one or more images of the subject via a radiation source and a radiation detector based at least in part on the imaging parameter; and
wherein the radiation detector receives electromagnetic rays transmitted though the subject by the radiation source while the radiation source travels along a path defined by a sweep angle that is less than 365 degrees.

11. The method of claim 10, wherein the sensor is an optical camera, and acquiring preliminary data regarding the subject via a sensor comprises:
acquiring an optical image of the subject via the optical camera.

12. The method of claim 10, wherein the sensor includes the radiation source and the radiation detector, and acquiring preliminary data regarding the subject via a sensor comprises:
acquiring a pre-shot of the subject via the radiation source and the radiation detector.

13. The method of claim 10, wherein the sensor is a depth camera, and acquiring preliminary data regarding the subject via a sensor comprises:
acquiring a depth image of the subject via the depth camera.

14. The method of claim 10, wherein the imaging parameter is at least one of a subject parameter, an acquisition parameter, a reconstruction parameter, and a display parameter.

15. The method of claim 10, wherein the path is configured for tomosynthesis.

16. The method of claim 10, wherein the electromagnetic rays are x-rays.

17. A non-transitory computer readable medium storing instructions configured to adapt a controller to:
acquire preliminary data regarding a subject via a sensor;
determine an imaging parameter from the preliminary data;
acquire one or more images of the subject via a radiation source and a radiation detector based at least in part on the imaging parameter; and
wherein the radiation detector receives electromagnetic rays transmitted though the subject by the radiation source while the radiation source travels along a path defined by a sweep angle that is less than 365 degrees.

18. The non-transitory computer readable medium of claim 17, wherein the sensor is an optical camera and the preliminary data is an optical image.

19. The non-transitory computer readable medium of claim 17, wherein the sensor includes the radiation source and the radiation detector and the preliminary data is a pre-shot.

20. The non-transitory computer readable medium of claim 17, wherein the imaging parameter is at least one of a subject parameter, an acquisition parameter, a reconstruction parameter, and a display parameter.
